(19) United States
(12) Patent Application Publication
(10) Pub. No.: US 2026/0224567 A1
(43) Pub. Date: Aug. 6, 2026

(54) METHODS FOR TREATING NEUROBLASTOMA WITH A DUAL ANAPLASTIC LYMPHOMA KINASE AND FOCAL ADHESION KINASE INHIBITOR

(71) Applicant: The Regents of the University of Michigan, Ann Arbor, MI (US)

(72) Inventors: Arul M. Chinnaiyan, Ann Arbor, MI (US); Seema Chugh, Ann Arbor, MI (US)

(21) Appl. No.: 19/159,155

(22) PCT Filed: Feb. 23, 2024
(86) PCT No.: PCT/US2024/017091

§ 371 Date: Aug. 22, 2025

Related U.S. Application Data
(60) Provisional application No. 63/447,762, filed on Feb. 23, 2023.

(51) Int. Cl. A61K 31/506; A61K 45/06; A61P 35/00; C12Q 1/6886

(52) U.S. Cl. CPC A61K 31/506; A61K 45/06; A61P 35/00; C12Q 1/6886; C12Q 2600/106

(57) ABSTRACT

Provided herein are methods for treating neuroblastoma characterized as having genetic aberrations in ALK, FAK, and/or MYCN with ESK440.

### CROSS-REFERENCE TO RELATED APPLICATION

The present invention claims the priority benefit of U.S. Provisional Patent Application No. 63/447,762 filed Feb. 23, 2023 which is incorporated by reference in its entirety.

### STATEMENT OF GOVERNMENTAL INTEREST

This invention was made with government support under CA231996 awarded by the National Institutes of Health. The government has certain rights in the invention.

### BACKGROUND

### Field

This disclosure provides methods for treating neuroblastoma (NB) and NB having genetic aberrations. In particular, provided herein are methods for treating neuroblastoma comprising administering a therapeutic agent, e.g., ESK440, capable of dual inhibition of ALK and FAK.

### Background

Neuroblastoma is the most common extracranial pediatric tumor and accounts for approximately 12% of cancer-related deaths in children. Maris N Engl J Med. 2010; 362(23):2202-11. While targeted therapies have been effective at treating the advanced states of several types of cancer, the development of targeted therapy for treating aggressive NB has yet to be realized. This is because N-myc proto-oncogene protein (MYCN) amplification, the most prevalent genetic aberration in aggressive NB, has been difficult to target. Huang et al. Cold Spring Harb Perspect Med. 2013; 3(10):a014415. In addition to MYCN amplification, approximately 10% of NB patients harbor genetic aberrations in ALK. These aberrations have been associated with poor survival, and the majority (85%) occur at three positions—R1275, F1174, and F1245. Mosse et al. Nature 2008; 455(7215):930-5; Bresler et al. Cancer Cell. 2014; 26(5):682-94. Studies have observed the co-occurrence of ALK mutations with MYCN amplifications and have suggested a synergistic effect on oncogenesis. Berry et al. Cancer Cell. 2012; 22(1):117-30; Zhu et al. Cancer Cell. 2012; 21(3):362-73.

ALK and FAK are multi-functional tyrosine kinases that play essential roles in neuronal development. Several genomic mutations of ALK, e.g., R1275, F1175, F1245, have been described in NB and other cancers. See, e.g., Della Corte et al., Mol Cancer 17, 30(2018). https://doi.org/10.1186/s12943-018-0776-2. FAK has also been associated with increased metastasis and is found to be overexpressed in MYCN-amplified NB. Beierle et al. Clin Cancer Res. 2008; 14(11):3299-305; Lee et al. Oncotarget. 2012; 3(12):1576-87.

The ALK inhibitor crizotinib has only shown modest responses in clinical studies of NB patients, and preclinical studies with crizotinib demonstrated reduced sensitivity in a NB model with F1174L-mutated ALK. Gandhi et al. Clin Cancer Res. 2012; 18(14):3737-42; Mosse et al., Lancet Oncol. 2013; 14(6):472-80. Crizotinib also has poor blood brain barrier penetration, one of the known challenges in targeting brain metastasis in relapsed NB. Costa et al. J Clin Oncol. 2011; 29(15):e443-5. Although other ALK inhibitors such as lorlatinib are being evaluated in clinical trials, the highly heterogeneous nature of the disease and development of resistance to ALK inhibitor therapies underscores the need for therapeutic strategies to treatment NB. Infarinato et al., Cancer Discov. 2016; 6(1):96-107; Zou et al. Cancer Cell. 2015; 28(1):70-81; Boeva et al. Nat Genet. 2017; 49(9):1408-13.

### BRIEF SUMMARY

In one aspect, the present disclosure provides a method of treating neuroblastoma in a subject in need thereof, the method comprising administering to the subject a therapeutically effective amount of(S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide (ESK440). In another embodiment, the neuroblastoma is characterized as having a genetic aberration, e.g. a mutation, overexpression, and/or amplification, of focal adhesion kinase (FAK), anaplastic lymphoma kinase (ALK), and/or N-myc proto-oncogene protein (MYCN).

In another aspect, the present disclosure provides a method of treating a subject having neuroblastoma, the method comprising:
(a) determining whether a genetic aberration, e.g., an ALK mutation, MYCN amplification, and/or FAK overexpression is present or absent in a biological sample taken from the subject; and
(b) administering a therapeutically effective amount of ESK440 to the subject if the genetic aberration is present in the biological sample.

In another aspect, the present disclosure provides a method, the method comprising administering a therapeutically effective amount of ESK440 to a subject in need thereof, wherein:
(a) the subject has neuroblastoma; and
(b) the neuroblastoma is characterized as having a genetic aberration, e.g., an ALK mutation, MYCN amplification, and/or FAK overexpression.

In another aspect, the present disclosure provides a method of identifying whether a subject having neuroblastoma as a candidate for treatment with ESK440, the method comprising:
(a) determining whether a genetic aberration, e.g., an ALK mutation, MYCN amplification, and/or FAK overexpression is present or absent in a biological sample taken from the subject; and
(b) identifying the subject as being a candidate for treatment if the genetic aberration is present; or
(c) identifying the subject as not being a candidate for treatment if the genetic aberration is absent.

### BRIEF DESCRIPTION OF DRAWINGS

FIG. 1 is a bar graph showing the one-week survival assay of diverse neuroblastoma (NB) preclinical models treated with ESK440.

FIG. 2 is a line graph showing the dose response for diverse NB cell lines treated with ESK440 for one week.

FIG. 3 a series of four Western blot experiments of ESK440 treated NB-1 cells showing ALK, FAK, ERK, MYCN, and H3 levels.

FIG. 4 is a bar graph showing the migration of ALK-amplified expression in ESK440-treated NB-1 cell lines.

FIG. 5 is a bar graph showing the invasion of ALK-amplified expression in ESK440-treated NB-1 cell lines.

FIG. 6 is a bar graph showing the migration of ALK wild-type (WT) expression in ESK440-treated IMR-32 cell lines.

FIG. 7 is a bar graph showing the invasion of ALK WT expression in ESK440-treated IMR-32 cell lines.

FIG. 8 is a series of four images showing the inhibition of phosphor-ALK in NB-1 cell line-derived xenograft tumors treated with vehicle or BID ESK440.

FIG. 9 is a series of four images showing the inhibition of phosphor-FAK in NB-1 cell line-derived xenograft tumors treated with vehicle or BID ESK440.

FIG. 10 is a series of two images showing the inhibition of MYCN in NB-1 cell line-derived xenograft tumors treated with vehicle or BID ESK440.

FIG. 11 is a pia chart of the overall MYCN response in vehicle- or ESK440-treated mice.

FIG. 12 is a line graph of the tumor volume of NB-1 cell line-derived xenograft tumors treated with either vehicle, ESK440 PO QD, or ESK440 PO BID.

FIG. 13 is a box and whisker plot of the tumor weight of NB-1 tumors after treatment with vehicle, ESK440 PO QD, or ESK440 PO BID.

FIG. 14 is a bar graph showing the tumor volume change from baseline of NB-1 tumors after treatment with vehicle, ESK440 PO QD, and ESK440 PO BID.

FIG. 15 is an image of tumors treated with vehicle, ESK440 PO QD, and ESK440 PO BID.

FIG. 16 is a series of six images of representative hematoxylin and eosin (H&E) (top) and Ki67 (bottom) stained NB-1 tumors treated with vehicle, ESK440 PO QD, or ESK440 PO BID.

FIG. 17 is a box and whisker plot of the Ki67-positive cells from NB-1 tumors treated with vehicle, ESK440 PO QD, or ESK440 PO BID.

FIG. 18 is a line graph of the change in body weight in NB-1 xenografted mice after treatment with either vehicle, ESK440 PO QD, or ESK440 PO BID.

FIG. 19 is a chart of blood chemistry results from NB tumor-bearing mice in vehicle, ESK440 PO QD, or ESK440 PO BID treatment groups.

FIG. 20 is a line graph of the tumor volume in NB patient-derived xenografts (PDX) of Felix (ALK F1254C mutation) tumors treated with vehicle, ESK440 PO QD, or ESK440 PO BID.

FIG. 21 is a bar graph of the tumor weight in NB PDX of Felix (ALK F1254C mutation) tumors treated with vehicle, ESK440 PO QD, or ESK440 PO BID.

FIG. 22 is a line graph of the body weight change in NB PDX of Felix (ALK F1254C mutation) tumors treated with vehicle, ESK440 PO QD, or ESK440 PO BID.

FIG. 23 is a line graph of the tumor volume in NB PDX of COG-N-452 (ALK F1174L mutation) tumors treated with vehicle, ESK440 PO QD, or ESK440 PO BID.

FIG. 24 is a bar graph of the tumor weight in NB PDX of COG-N-452 (ALK F1174L mutation) tumors treated with vehicle, ESK440 PO QD, or ESK440 PO BID.

FIG. 25 is a line graph of the body weight change in NB PDX of COG-N-452 (ALK F1174L mutation) tumors treated with vehicle, ESK440 PO QD, or ESK440 PO BID.

FIG. 26 is a line graph of the tumor volume in NB PDX of COG-N-453 (ALK F1174L mutation) tumors treated with vehicle or varying doses of ESK440 PO BID.

FIG. 27 is a bar graph of the tumor weight in NB PDX of COG-N-453 (ALK F1174L mutation) tumors treated with vehicle or varying doses of ESK440 PO BID

FIG. 28 is a line graph of the body weight change in NB PDX of COG-N-453 (ALK F1174L mutation) tumors treated with vehicle or varying doses of ESK440 PO BID

FIG. 29 is a line graph of the dose response for NB-1643 cell line treated with ESK440 or lorlatinib.

FIG. 30 is a line graph of the dose response for CHLA-20 cell line treated with ESK440 or lorlatinib.

FIG. 31 is a line graph of the dose response for COG-N-529 cell line treated with ESK440 or lorlatinib.

FIG. 32 is a line graph of the viability of lorlatinib-resistant (LR) and COG-561 (parental) cell lines.

FIG. 33 is a line graph of the dose response for LR cells treated with ESK440 or lorlatinib.

### DETAILED DESCRIPTION

In one embodiment, the present disclosure provides a method of treating neuroblastoma in a subject in need thereof, the method comprising administering to the subject a therapeutically effective amount of ESK440.

In another embodiment, the neuroblastoma is characterized as having a genetic aberration, e.g. a mutation, overexpression, and/or amplification, of focal adhesion kinase (FAK), anaplastic lymphoma kinase (ALK), and/or N-myc proto-oncogene protein (MYCN).

In another embodiment, the neuroblastoma is an ALK-mutated neuroblastoma, a MYCN-amplified neuroblastoma, and/or a FAK-overexpressed neuroblastoma.

In another embodiment, the neuroblastoma is an ALK-mutated neuroblastoma.

In another embodiment, the neuroblastoma is an ALK-mutated neuroblastoma comprising a F1275 mutation, an ALK F1245 mutation, or an ALK F1174 mutation.

In another embodiment, the neuroblastoma is a MYCN-amplified neuroblastoma.

In another embodiment, the neuroblastoma is a FAK-overexpressed neuroblastoma.

In another embodiment, the subject is aged 2 years to less than 12 years.

In another embodiment, the subject is aged 12 years to less than 22 years.

In another embodiment, the subject is aged 22 years or more.

In another embodiment, one or more anticancer agents are administered to the subject in combination, i.e., co-administered, with ESK440. In another embodiment, the anticancer agent is one or more of a chemotherapeutic agent, an immune checkpoint inhibitor, e.g., pembrolizumab, nivolumab, cemipilimab, atezolizumab, avelumab, durvalumab, ipilimumab, or radiation therapy.

A number of suitable anticancer agents are contemplated for use in the methods of the present disclosure. Indeed, the present disclosure contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); antiestrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present disclosure provide ESK440 and at least one anti-hyperproliferative or antineoplastic agent selected from alkylating agents, antimetabolites, and natural products, e.g., herbs and other plant and/or animal derived compounds.

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3)alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide); and 17) gonadotropin-releasing hormone analogs (e.g., leuprolide).

Any oncolytic agent that is routinely used in a cancer therapy context finds use in the compositions and methods of the present disclosure. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and ILEX |
| (IgG1κ anti CD52 antibody) | | Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4 H-pyrazolo[3,4-d]pyrimidin-4- | | Research Triangle Park, |
| one monosodium salt) | | NC |
| Altretamine | Hexalen | US Bioscience, West |
| (N,N,N′,N′,N″,N″,-hexamethyl-1,3,5-triazine- | | Conshohocken, PA |
| 2, 4, 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a, a, a′, a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, |
| (lyophilized preparation of an attenuated strain | | Corp., Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl) ethenyl] benzoic | | |
| acid) | | |
| bexarotene gel | Targretin | Ligand Pharmaceuticals |
| Bleomycin | Blenoxane | Bristol-Myers Squibb |
| (cytotoxic glycopeptide antibiotics produced | | Co., NY, NY |
| by Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0, 0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, 2- | R.W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, | Bristol-Myers Squibb |
| (2-[bis(2-chloroethyl)amino] tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, |
| | | Inc., San Diego, CA |
| Dacarbazine | DTIC-Dome | Bayer AG, Leverkusen, |
| (5-(3,3-dimethyl-1-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | Danuo Xome | Nexstar |
| ((8S-cis)-8-acetyl-10-[(3-amino-2,3,6- | | Pharmaceuticals, Inc., |
| trideoxy-á-L-lyxo-hexopyranosyl)oxy]- | | Boulder, CO |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, |
| ((1 S,3 S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)- | | |
| L-lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b, 13a-hexahydroxytax-11-en-9- | | |
| one 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin | Pharmacia & Upjohn |
| | PFS Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott′s B Solution | Elliott′s B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3, 17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, |
| (5-fluoro-2,4(1H,3H)-pyrimidinedione) | | Inc., Humacao, Puerto |
| | | Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5-(10)- | | |
| triene-3,17-beta-diol) | | |
| Gemcitabine | Gemzar | Eli Lilly |
| (2′-deoxy-2′, 2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| | | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3- | | |
| (p-isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5, 12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′, 4′: | | |
| 6,7] indolizino[1,2-b] quinoline-3,14(4H, 12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((-)-(S)-2,3,5, 6-tetrahydro-6-phenylimidazo | | Foundation, Titusville, |
| [2,1-b] thiazole monohydrochloride | | NJ |
| C₁₁H₁₂N₂S•HCl) | | |
| Lomustine | CeeNU | Bristol-Myers Squibb |
| (1-(2-chloro-ethyl)-3-cyclohexyl-1- | | |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L- | | |
| phenylalanine) | | |
| Mercaptopurine, 6-MP | Purinethol | GlaxoSmithKline |
| (1,7-dihydro-6 H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7- | | Exton, Pa |
| one) | | |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′] | | NY, NY |
| [oxalato(2-)-O,O′] platinum) | | |
| Paclitaxel | TAXOL | Bristol-Myers Squibb |
| (5ß, 20-Epoxy-1,2a, 4,7ß, 10ß, 13a- | | |
| hexahydroxytax-11-en-9-one 4, 10-diacetate 2- | | |
| benzoate 13-ester with (2R, 3 S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl) 11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South |
| (recombinant anti-CD20 antibody) | | San Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]- | | |
| a(and b)-D-glucopyranose and 220 mg | | |
| citric acid anhydrous) | | |
| Talc | Sclerosol | Bryan, Corp., Woburn, |
| (Mg₃Si₄O₁₀ (OH)₂) | | MA |
| Tamoxifen | Nolvadex | AstraZeneca |
| ((Z)2-[4-(1,2-diphenyl-1-butenyl) phenoxy]-N, | | Pharmaceuticals |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]- | | |
| as-tetrazine-8-carboxamide) | | |
| teniposide, VM-26 | Vumon | Bristol-Myers Squibb |
| (4′-demethylepipodophyllotoxin 9-[4,6-0-(R)- | | |
| 2-thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmith Kline |
| (2-amino-1,7-dihydro-6 H-purine-6-thione) | | |
| Thiotepa | Thioplex | Immunex Corporation |
| (Aziridine, 1,1′,1″-phosphinothioylidynetris-, | | |
| or Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′, 4′: 6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra--> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′ ,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present disclosure provides methods for administering the ESK440 with radiation therapy. The disclosure is not limited by the types, amounts, or delivery and administration systems used to deliver the therapeutic dose of radiation to the subject. For example, the subject may receive photon radiotherapy, particle beam radiation therapy, other types of radiotherapies, and combinations thereof. In some embodiments, the radiation is delivered to the subject using a linear accelerator. In still other embodiments, the radiation is delivered using a gamma knife.

The source of radiation can be external or internal to the subject. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by subjects. Internal radiation therapy involves implanting a radiation-emitting source, such as beads, wires, pellets, capsules, particles, and the like, inside the body at or near the tumor site including the use of delivery systems that specifically target cancer cells, e.g., using particles attached to cancer cell binding ligands. Such implants can be removed following treatment, or left in the body inactive. Types of internal radiation therapy include, but are not limited to, brachytherapy, interstitial irradiation, intracavity irradiation, radioimmunotherapy, and the like.

The subject may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an subject, so long as the dose of radiation is tolerated by the subject without unacceptable negative side-effects. Suitable types of radiotherapy include, for example, ionizing (electromagnetic) radiotherapy, e.g., X-rays or gamma rays, or particle beam radiation therapy, e.g., high linear energy radiation. Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to s subject is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present disclosure.

In some embodiments of the present disclosure, ESK440 and one or more anticancer agents are administered to a subject under one or more of the following conditions: at different periodicities, at different durations, at different concentrations, by different administration routes, etc. In some embodiments, ESK440 is administered prior to the anticancer agent, e.g., 0.5, 1, 2, 3, 4, 5, 10, 12, or 18 hours, 1, 2, 3, 4, 5, or 6 days, or 1, 2, 3, or 4 weeks prior to the administration of anticancer agent. In some embodiments, ESK440 is administered after the anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, ESK440 and the anticancer agent are administered concurrently but on different schedules, e.g., the compound is administered daily while the therapeutic or anticancer agent is administered once a week, once every two weeks, once every three weeks, or once every four weeks. In other embodiments, ESK440 administered once a week while the anticancer agent is administered daily, once a week, once every two weeks, once every three weeks, or once every four weeks.

Compositions within the scope of this disclosure include all compositions wherein ESK440 is contained in an amount which is effective to achieve its intended purpose. While individual needs vary, determination of optimal ranges of effective amounts of each component is within the skill of the art. Typically, ESK440 may be administered to subjects, e.g., human cancer patients, orally at a dose of 0.0025 to 100 mg/kg, or an equivalent amount of the pharmaceutically acceptable salt thereof, per day of the body weight of the mammal being treated for disorders responsive to induction of apoptosis. In one embodiment, about 0.01 to about 25 mg/kg of ESK440 is orally administered to treat, ameliorate, or prevent neuroblastoma. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of ESK440. The unit dose may be administered one or more times daily as one or more tablets or capsules each containing from about 0.1 to about 10 mg, conveniently about 0.25 to 50 mg of ESK440.

In a topical formulation, ESK440 may be present at a concentration of about 0.01 to 100 mg per gram of carrier. In a one embodiment, ESK440 is present at a concentration of about 0.07-1.0 mg/ml, for example, about 0.1-0.5 mg/ml, and in one embodiment, about 0.4 mg/ml.

In addition to administering ESK440 as a raw chemical, the ESK440 may be administered as part of a pharmaceutical formulation containing suitable pharmaceutically acceptable carriers comprising excipients and auxiliaries which facilitate processing of ESK440 into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of ESK440, together with the excipient.

The pharmaceutical compositions comprising ESK440 may be administered to any subject which may experience the beneficial effects of ESK440. Foremost among such subjects are mammals, e.g., humans, although the disclosure is not intended to be so limited. Other subjects include veterinary animals (cows, sheep, pigs, horses, dogs, cats and the like).

ESK440 and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present disclosure are manufactured in a manner which is itself known, for example, by means of conventional mixing, granulating, dragee-making, dissolving, or lyophilizing processes. Thus, pharmaceutical preparations for oral use can be obtained by combining ESK440 with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this disclosure are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

In another embodiment, present disclosure provides methods of treating a subject, e.g., a human patient, having cancer, e.g., neuroblastoma, comprising (a) determining whether a biomarker is present or absent in a biological sample taken from the subject; and (b) administering a therapeutically effective amount of ESK440 and, optionally, one or more additional anticancer agent to the subject if the biomarker is present in the biological sample. See, e.g., Goossens et al., Transl Cancer Res. 4:256-269(2015); Kamel and Al-Amodi, Genomics Proteomics Bioinformatics 15:220-235(2017); and Konikova and Kusenda, Neoplasma 50:31-40(2003).

Biomarkers include, but are not limited to, genetic aberrations, e.g., FAK, ALK, and/or MYCN overexpression, mutation, and/or amplification, in cancer, e.g., neuroblastoma. In one embodiment, the measurable aspect of the biomarker, e.g., FAK, is its overexpression status. In another embodiment, the measurable aspect of the biomarker, e.g., ALK, is its mutation status. In another embodiment, the measurable aspect of the biomarker, e.g., MYCN, is its amplification status.

In another embodiment, the biomarker is a genetic aberration of ALK, MYCN and/or FAK, which, collectively, is differentially present in a subject of one phenotypic status, e.g., a subject having neuroblastoma, as compared with another phenotypic status, e.g., a normal undiseased subject.

In one embodiment, the biomarker is FAK overexpression, which is differentially present in a subject of one phenotypic status, e.g., a subject having neuroblastoma, as compared with another phenotypic status, e.g., a normal undiseased subject or a subject having neuroblastoma without overexpression of FAK.

In another embodiment, the biomarker is an ALK mutation, which is differentially present in a subject of one phenotypic status, e.g., a subject having neuroblastoma, as compared with another phenotypic status, e.g., a normal undiseased subject or a patient having neuroblastoma without an ALK mutation.

In another embodiment, the biomarker is MYCN amplification, which is differentially present in a subject of one phenotypic status, e.g., a subject having neuroblastoma, as compared with another phenotypic status, e.g., a normal undiseased subject or a patient having neuroblastoma without MYCN amplifcation.

Biomarker standards can be predetermined, determined concurrently, or determined after a biological sample is obtained from the subject. Biomarker standards for use with the methods described herein can, for example, include data from samples from subjects without cancer, e.g., neuroblastoma; data from samples from subjects with cancer. Comparisons can be made to establish predetermined threshold biomarker standards for different classes of subjects, e.g., diseased vs. non-diseased subjects. The standards can be run in the same assay or can be known standards from a previous assay.

A biomarker is differentially present between different phenotypic status groups if the mean or median expression or mutation or amplification levels of the biomarker is calculated to be different, i.e., higher or lower, between the groups. Thus, biomarkers provide an indication that a subject, e.g., a cancer patient, belongs to one phenotypic status or another.

The determination of the overexpression level or mutation status or amplification of a biomarker in a patient can be performed using any of the many methods known in the art. Any method known in the art for quantitating specific proteins and/or detecting FAK, ALK, and/or MYCN overexpression, mutations, and/or amplifications, respectively, or the overexpression, mutation, and/or amplification levels of any other biomarker in a patient or a biological sample may be used in the methods of the disclosure. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al. Cancer 83:1401 (1998); Hudlebusch et al., Clin Cancer Res 17:2919-2933(2011). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, NY, (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from the tumor tissue sample using RNAse free techniques. Such techniques are commonly known in the art.

In one embodiment of the disclosure, a biological sample is obtained from the subject and the biological sample is assayed for determination of biomarker expression or biomarker mutation status or biomarker amplification status.

In another embodiment, the present disclosure provides a method of treating a subject having neuroblastoma, the method comprising:
(a) determining whether a genetic aberration, e.g., an ALK mutation, MYCN amplification, and/or FAK overexpression is present or absent in a biological sample taken from the subject; and
(b) administering a therapeutically effective amount of ESK440 to the subject if the genetic aberration overexpression is present in the biological sample.

In another embodiment, the present disclosure provides a method, comprising administering a therapeutically effective amount of ESK440 to a subject in need thereof, wherein:
(a) the subject has neuroblastoma; and
(b) the neuroblasoma is characterized as having a genetic aberration, e.g., an ALK mutation, MYCN amplification, and/or FAK overexpression.

In another embodiment, the neuroblasoma is characterized as having an ALK mutation.

In another embodiment, the neuroblasoma is characterized as having MYCN amplification.

In another embodiment, the neuroblasoma is characterized as having FAK overexperession.

In another embodiment, the present disclosure provides a method of identifying whether a subject having neuroblasoma is a candidate for treatment with ESK440, the method comprising:
(a) determining whether a genetic aberration, e.g., an ALK mutation, MYCN amplification, and/or FAK overexpression is present or absent in a biological sample taken from the subject; and
(b) identifying the subject as being a candidate for treatment if the genetic aberration is present; or
(c) identifying the subject as not being a candidate for treatment if the genetic aberration is absent.

In another embodiment, the present disclosure provides a method of predicting treatment outcome in a subject having neuroblasoma, the method comprising determining whether a genetic aberration, e.g., an ALK mutation, MYCN amplification, and/or FAK overexpression is present or absent in a biological sample taken from the subject, wherein:
(a) the presence of the genetic aberration in the biological sample indicates that administering ESK440 to the subject will likely cause a favorable therapeutic response; and
(b) the absence of the genetic aberration in the biological sample indicates that administering ESK440 to the subject will likely cause an unfavorable therapeutic response.

The term “ESK440” refers to(S)-2-((5-chloro-2-((6-(4-(2-hydroxyethyl) piperazin-1-yl)-1-methoxy-6,7,8,9-tetrahydro-5H-benzo[7]annulen-2-yl)amino)pyrimidin-4-yl)amino)-N-methylbenzamide:

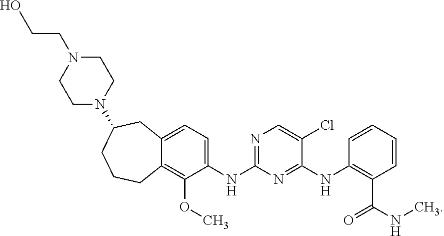 [SMILES: CNC(=O)c1ccccc1Nc1nc(Nc2ccc3c(c2OC)CCC[C@H](N2CCN(CCO)CC2)C3)ncc1Cl]

ESK440 (formerly known as CEP37440) inhibits the enzymatic and cellular biochemical activity of both ALK and FAK at low nanomolar concentrations. See, e.g., Ott et al., J Med Chem 2016; 59(16):7478-7496. ESK440 penetrates the blood brain barrier.

The term “anticancer agent” as used herein, refer to any therapeutic agent, e.g., chemotherapeutic compounds and/or molecular therapeutic compounds, antisense therapies, radiation therapies, or surgical interventions, used in the treatment of hyperproliferative diseases such as cancer, e.g., in mammals, e.g., in humans.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of neuroblasoma, in one embodiment, a therapeutically effective amount will refer to the amount of a therapeutic agent that decreases the rate of tumor growth, decreases tumor mass, decreases the number of metastases, increases time to tumor progression, or increases survival time by at least 1%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “neoplastic disease,” as used herein, refers to any abnormal growth of cells being either benign (non-cancerous) or malignant (cancerous).

The term “normal cell,” as used herein, refers to a cell that is not undergoing abnormal growth or division. Normal cells are non-cancerous and are not part of any hyperproliferative disease or disorder.

The term “anti-neoplastic agent,” as used herein, refers to any compound that retards the proliferation, growth, or spread of a targeted (e.g., malignant) neoplasm.

The term “ALK-mutated neuroblastoma” and the like as used herein refers to a neuroblastoma, e.g., a neuroblastoma tumor or tissue, comprising mutated ALK. Mutations in three positions—R1275, F1174, and F1245—account for around 85% of ALK mutations in NB and are hotspots for several lower frequency mutations. See, e.g., Trigg et al., Cancers (Basel) 2018; 10(4) 113.

The term “FAK-overexpressed neuroblastoma” and the like as used herein refers to a neuroblastoma, e.g., a neuroblastoma tumor or tissue, comprising an overexpression of FAK. See, e.g., Lee et al., Oncotarget. 2012; 3(12):1576-1587.

The term “MYCN-amplified neuroblastoma” and the like as used herein refers to to a neuroblastoma, e.g., a neuroblastoma tumor or tissue, comprising an amplification of MYCN. See, e.g., Dzieran et al., PNAS 2018: 115(6) E1229-1238.

The terms “a” and “an” refer to one or more.

The term “about,” as used herein, includes the recited number+10%. Thus, “about 10” means 9 to 11.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of ESK440 to a subject in need of such treatment.

Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The terms “prevent,” “preventing,” and “prevention,” as used herein, refer to a decrease in the occurrence of pathological cells, e.g., hyperproliferative or neoplastic cells, in an subject. The prevention may be complete, e.g., the total absence of pathological cells in a subject. The prevention may also be partial, such that the occurrence of pathological cells in a subject is less than that which would have occurred without the present disclosure.

The term “biological sample” as used herein refers any tissue or fluid from a subject that is suitable for detecting a biomarker, e.g., a genetic aberration. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for genetic aberrations using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (clg-FISH). A biological sample can be obtained using techniques that are well within the scope of ordinary knowledge of a clinical practioner. In one embodiment of the disclosure, the biological sample comprises blood cells.

The phrase “in combination” as used in connection with the administration ESK440 and one or more additional anticancer agents to a subject means that the ESK440 and one or more additional anticancer agents can be administered to the subject together, e.g., as part of a single pharmaceutical composition or formulation, or separately, e.g., as part of two or more separate pharmaceutical compositions or formulations. The phrase “in combination” as used in connection with the administration of ESK440 and one or more additional anticancer agents to a subject is thus intended to embrace administration of ESK440 and one or more additional anticancer agents in a sequential manner, wherein ESK440 and one or more additional anticancer agents are administered to the subject at a different time, as well as administration concurrently, or in a substantially simultaneous manner. Simultaneous administration can be accomplished, for example, by administering to the subject a single capsule having a fixed ratio of each of ESK440 and the one or more additional anticancer agents or in multiple, single capsules, tablets, etc. for each of ESK440 and the one or more additional anticancer agents. Sequential or substantially simultaneous administration of the ESK440 and the one or more additional anticancer agents can be accomplished by any appropriate route including, but not limited to, oral routes, intravenous routes, intramuscular routes, and direct absorption through mucous membrane tissues. ESK440 and the one or more additional anticancer agents can be administered by the same route or by different routes. For example, an anticancer agent of the combination may be administered by intravenous injection while ESK440 of the combination may be administered orally. Alternatively, for example, both ESK440 and the one or more additional anticancer agents may be administered orally or both ESK440 and the one or more additional anticancer agents may be administered by intravenous injection. ESK440 and one or more additional anticancer agents may also be administered in alternation. In one embodiment, ESK440 and the one or more additional anticancer agents are administered to a subject separately, e.g., as part of two or more separate pharmaceutical compositions or formulations. The same principles apply when a ESK440 and two or more anticancer agents are administered in combination to a subject.

### EXAMPLES

The following examples are illustrative, but not limiting, of the compounds, compositions, and methods of the present disclosure. Other suitable modifications and adaptations of the variety of conditions and parameters normally encountered in clinical therapy and which are obvious to those skilled in the art are within the spirit and scope of the disclosure.

### Example 1

### ESK440 Inhibits Neuroblastoma Growth In Vitro

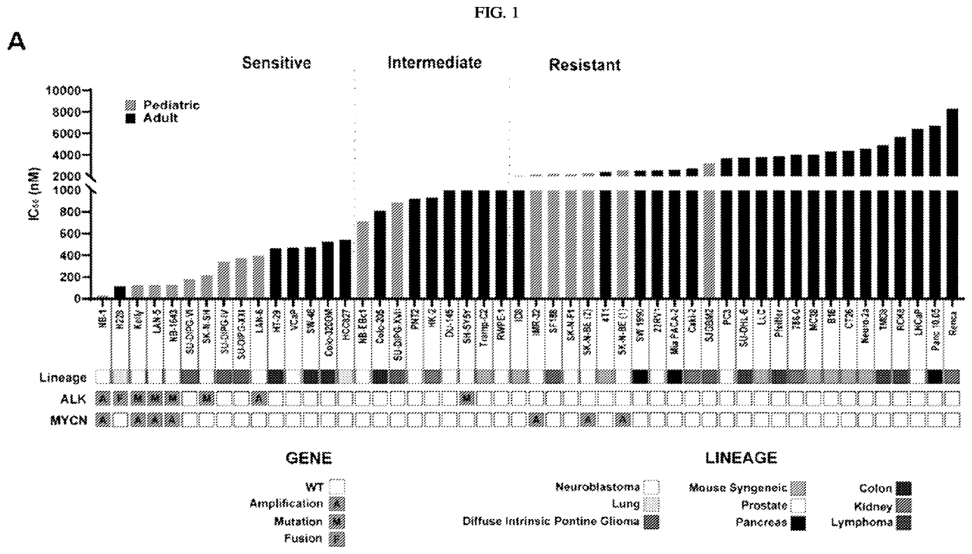

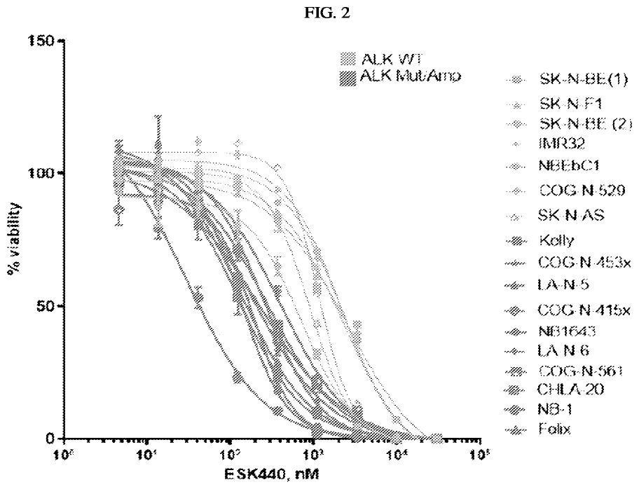

The efficacy of ESK440 in preventing cancer cell growth was analyzed in an extensive panel of cell lines from several different lineages (FIG. 1). These cell lines were treated with increasing concentrations of ESK440 for one week, and the half-maximum inhibitory concentration (IC₅₀) values for ESK440 were calculated and graphed for each cell line. As shown in FIG. 1 and Table 1, NB cell lines with ALK genomic aberrations were surprisingly sensitive to ESK440 treatment. The individual dose curves for each of the NB cell lines are shown in FIG. 2.

| | Cell Line | ALK status | MYCN status | IC₅₀ (nM) |
|---|---|---|---|---|
| 1 | NB-1 | Amp | Amp | 29.34 |
| 2 | Kelly | F1174L | Amp | 128.3 |
| 3 | LAN-5 | R1275Q | Amp | 130 |
| 4 | NB-1643 | R1275Q | Amp | 132.1 |
| 5 | COG-N-453x | ALKF1174L | Amp | 172.9 |
| 6 | COG-N-415x | F1174L | Amp | 188.7 |
| 7 | COG-N-561 | F1245I | Amp | 276.3 |
| 8 | CHLA-20 | R1275Q | Non-Amp | 225 |
| 9 | Felix | F1245V | Non-Amp | 252.4 |
| 10 | LAN-6 | Amp | Non-Amp | 396.5 |
| 11 | NB-EBc1 | WT | Non-Amp | 716.7 |
| 12 | SK-N-AS | WT | Non-Amp | 910.9 |
| 13 | COG-N-529 | WT | Non-Amp | 1150 |
| 14 | IMR-32 | WT | Amp | 2179 |
| 15 | SK-N-BE (2) | WT | Amp | 2277 |
| 16 | SK-N-BE (1) | WT | Amp | 2521 |

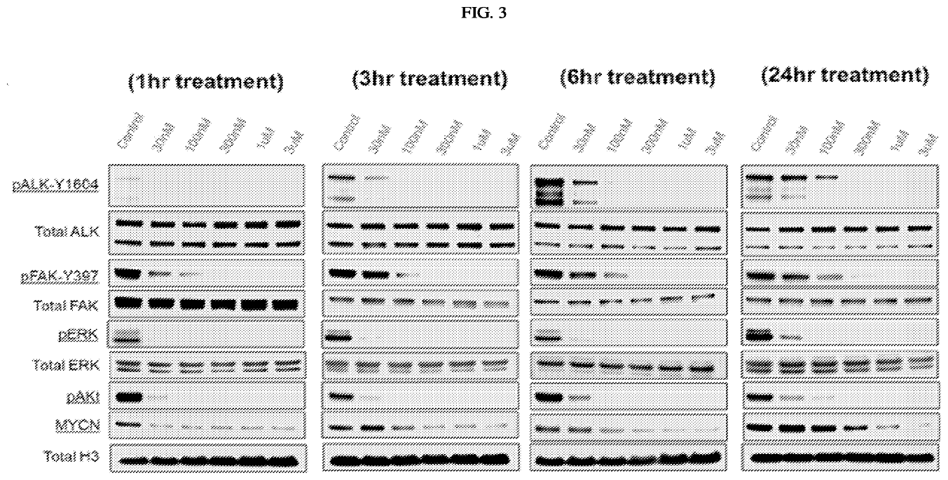

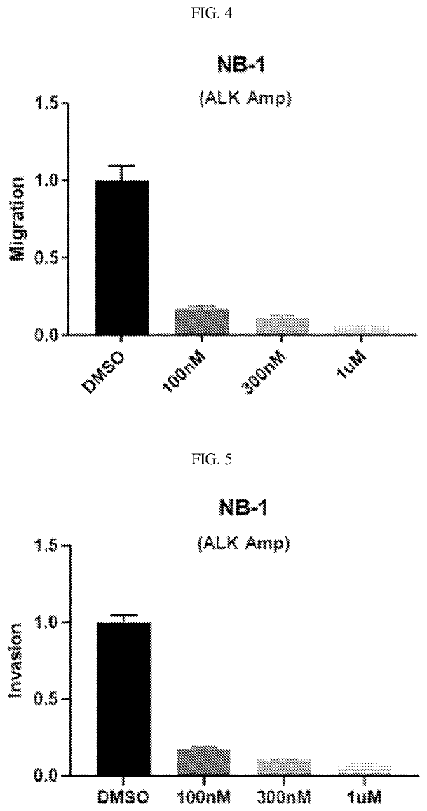

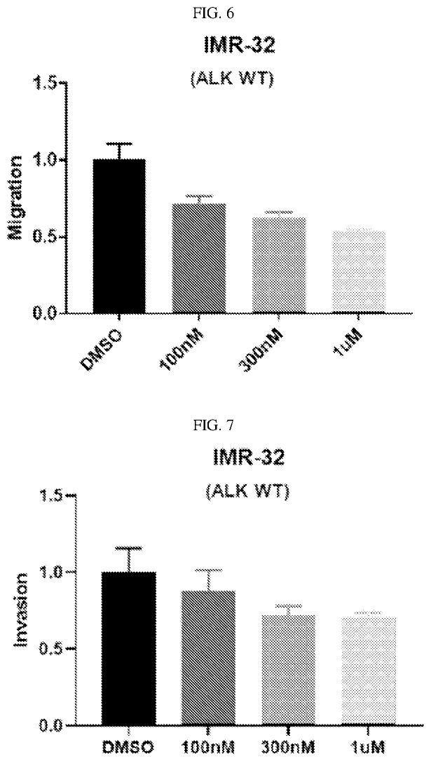

The functional impact of ESK440 in NB cells was also tested. NB-1 cells were treated with increasing concentrations of ESK440 for one, three, six, and 24 hours. Cell lysates were then harvested and analyzed by western blot for levels of ALK, FAK, and associated downstream targets. As shown in FIG. 3, ESK440 treatment rapidly diminished levels of phosphorylated ALK, FAK, ERK, and AKT. These levels remained decreased over the duration of the experiment. ESK440's inhibition of ALK/FAK also showed a downstream decrease in MYCN protein levels. As these signaling pathways impinge upon cell motility and invasion, Dawson et al. Nat Rev Cancer. 2021; 21(5):313-24. PMCID: PMC8276817, transwell migration and invasion assays were conducted with ESK440 in ALK-amplified NB-1 cells and compared to ALK wild-type IMR-32 cells. Treatment of NB-1 cells with ESK440 dramatically reduced cellular migration and invasion, while cells without ALK amplification, IMR-32 cells were not significantly affected (FIGS. 4-7).

### Example 2

### ESK440 Inhibits Neuroblastoma Cancer Growth In Vivo.

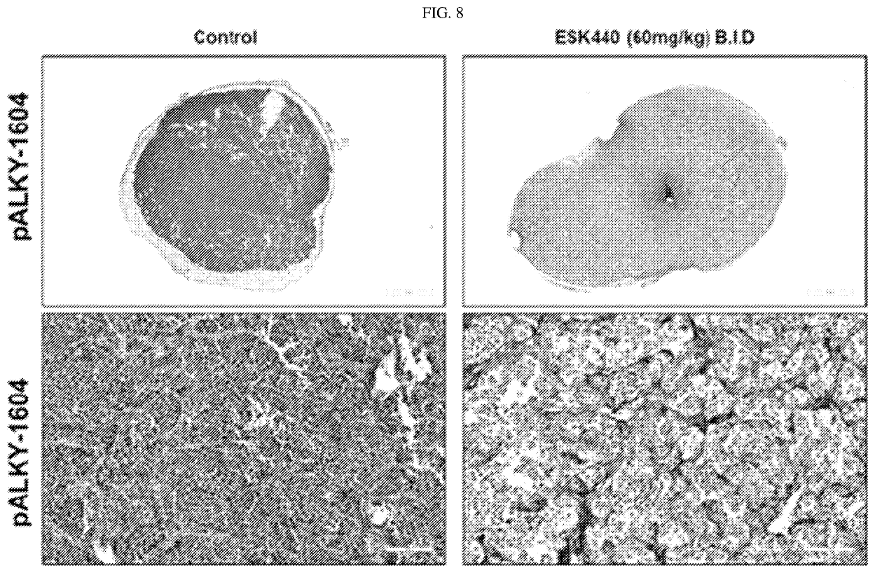

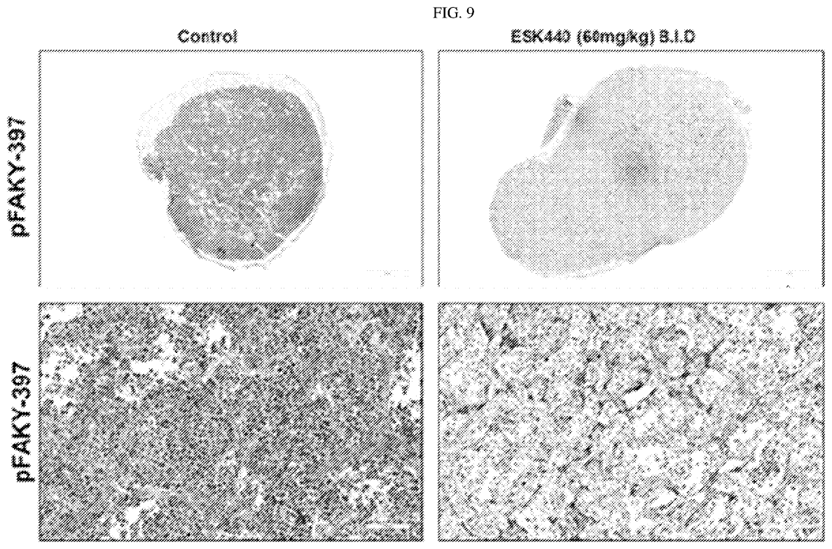

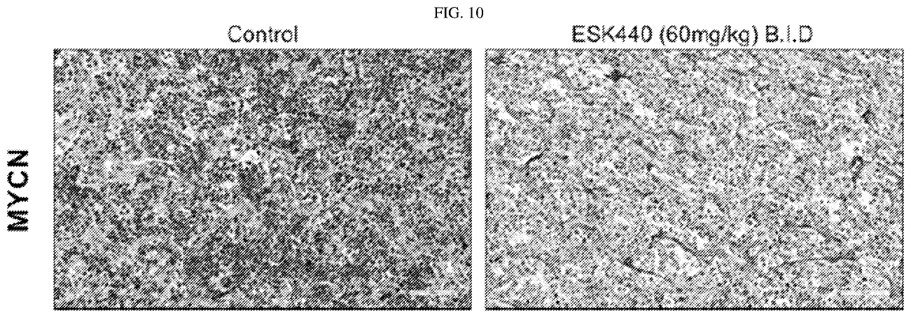

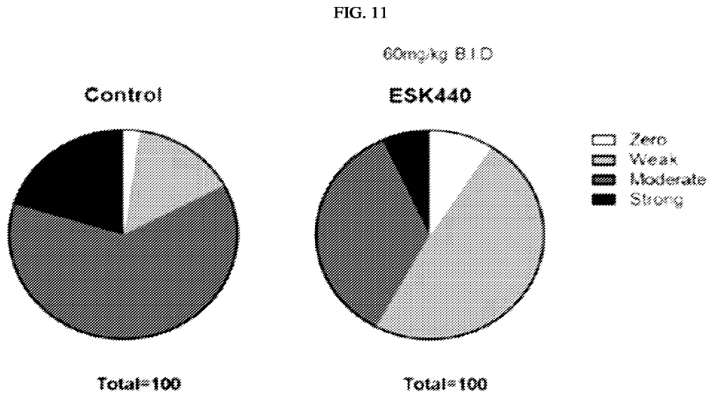

The combined in vitro data of EXAMPLE 1 showing inhibition of cell growth, migration, and invasion suggested that ESK440 may have activity as an anti-cancer agent in NB tumors harboring ALK genomic aberrations. To test this, several experiments in mice bearing cell line- or patient-derived xenografts (PDXs) were undertaken. NB-1 cell line xenografts were established subcutaneously into non-castrated mice until each tumor reached an average size of 100 mm³, after which mice were randomized into two groups that received either vehicle or 60 mg/kg BID ESK440 five days per week, once per day by oral gavage. Pharmacodynamic (PD) assessment of ESK440 treatment (60 mg/kg BID) at one week in NB-1 xenografts showed that ESK440 achieved on-target inhibition of ALK (FIG. 8) and FAK (FIG. 9) activity and also decreased levels of MYCN in vivo (FIGS. 10 and 11).

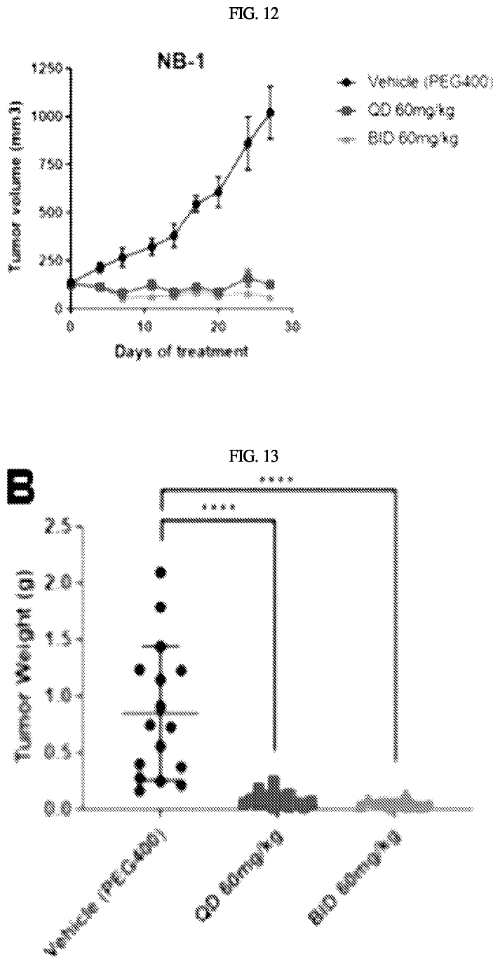

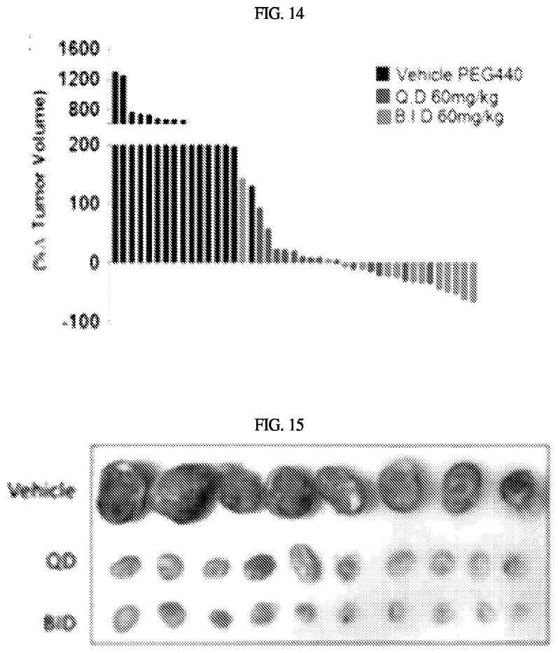

For tumor growth experiments, CB17 severe combined immunodeficiency (SCID) mice were injected with 5×10⁶ NB-1 cells per site and developed until tumors reached an average size of 100 mm³, after which mice were randomized into two groups. and treated with vehicle (PEG400, QD), ESK440(60 mg/kg) PO QD, or ESK440 (60 mg/kg) PO BID for five days per week for four weeks. Measurement of tumor volume, monitored twice per week, over the experiment showed that both ESK440 dosing regimens resulted in strong inhibition of tumor growth and end-point tumor weight (FIG. 12-15).

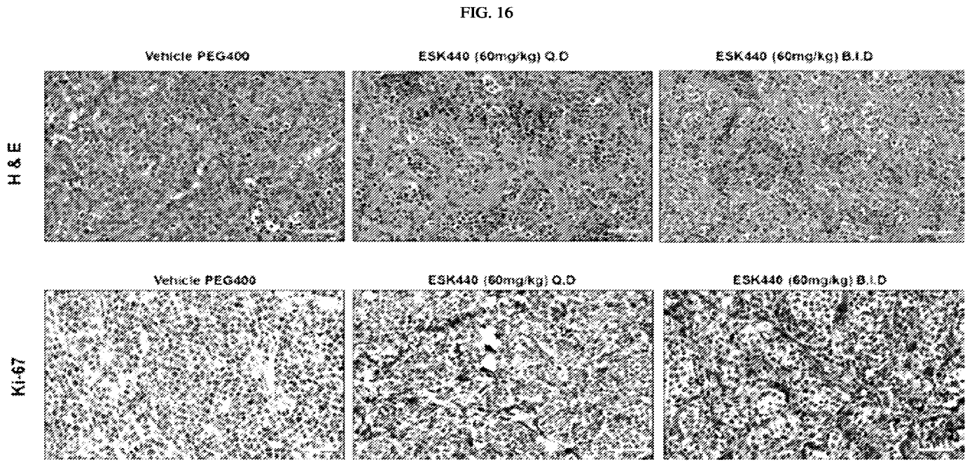

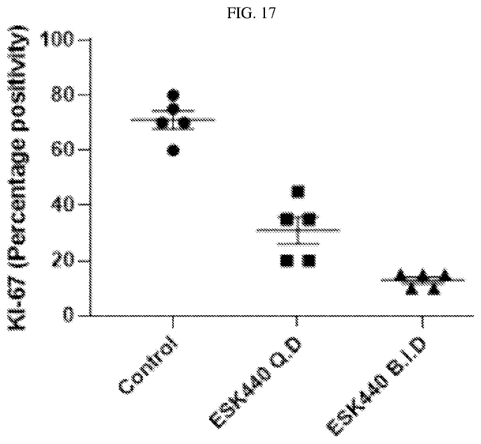

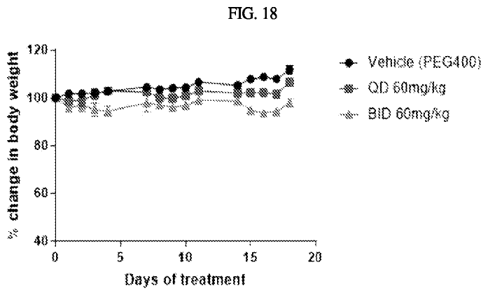

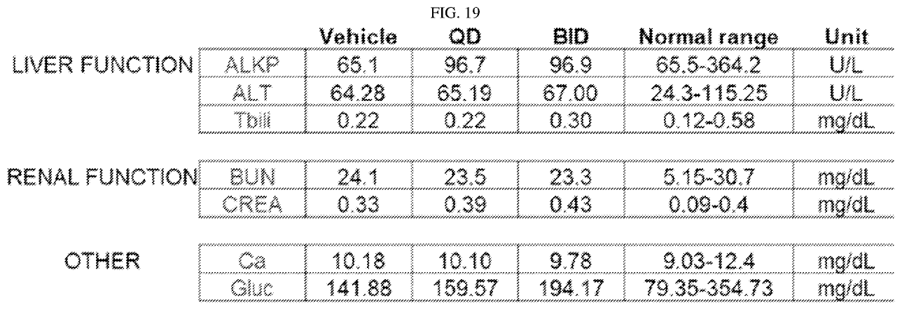

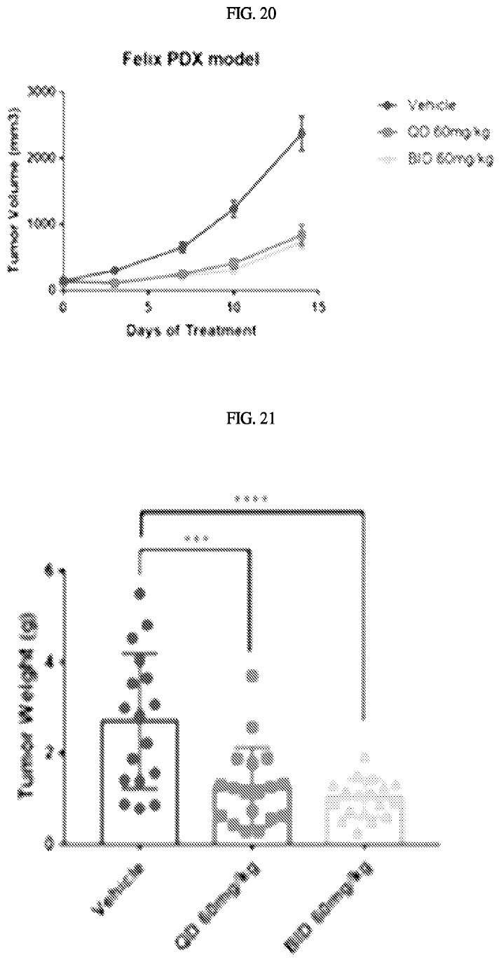

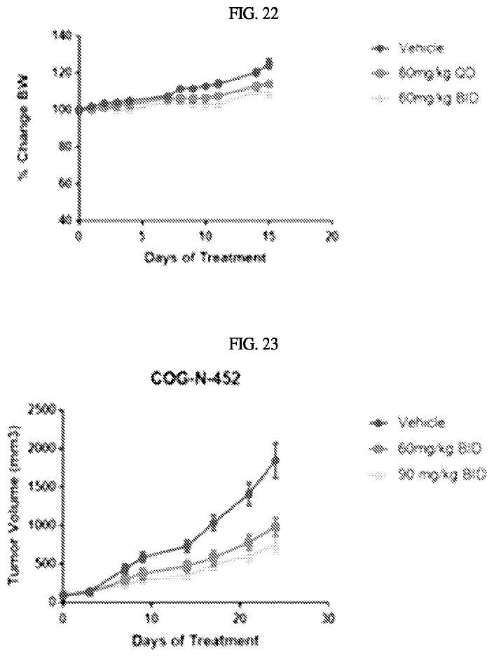

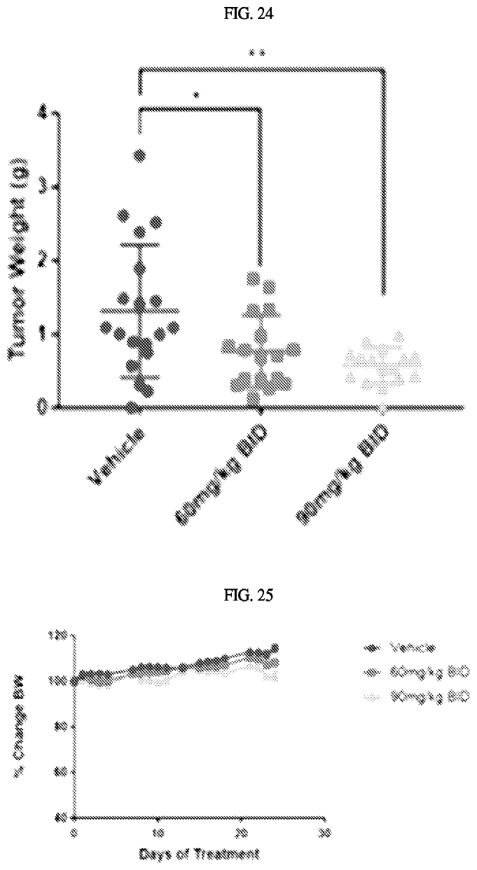

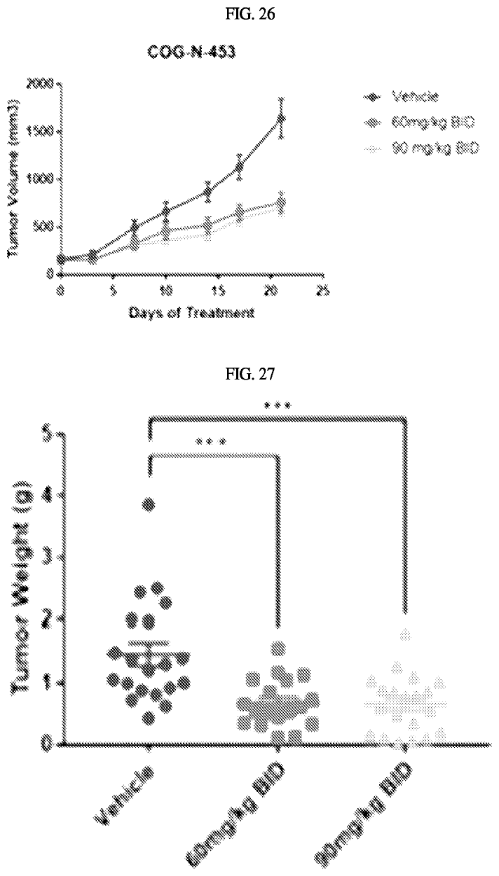

Immunohistochemistry (IHC) for the Ki67 proliferation marker similarly showed that ESK440 markedly decreased the percentage of proliferating cells in tumors (FIGS. 16 and 17). Importantly, body weights of mice remained stable with ESK440 treatment compared to vehicle (FIG. 17), and blood chemistry analysis showed no damage to liver or renal functioning (FIG. 18). The efficacy of ESK440 was also examined in three NB PDX models with ALK mutations-Felix (ALK F1254C mutation), COG-N-452 (ALK F1174L mutation), and COG-N-453 (ALK F1174L mutation). In each PDX, ESK440 treatment decreased tumor growth and weight while not affecting the body weights of mice (FIGS. 19-27). Altogether, in four in vivo models of NB, ESK440 decreased tumor growth while displaying no signs of toxicity.

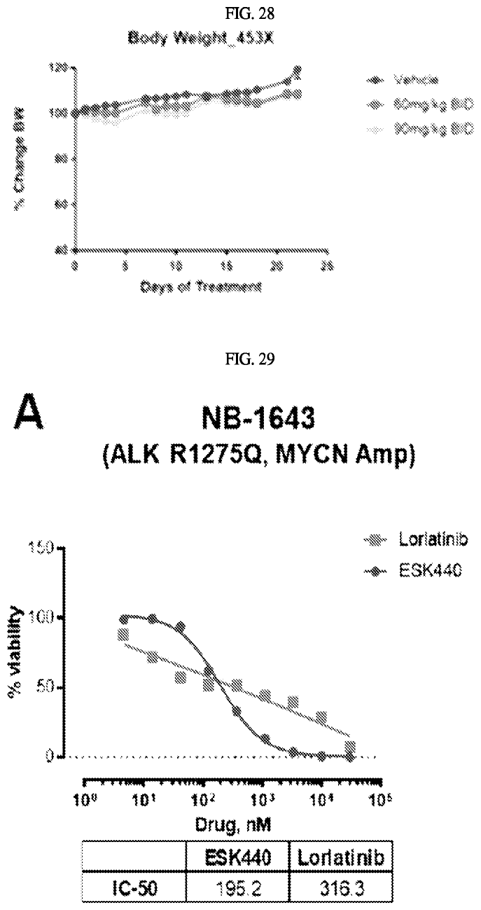

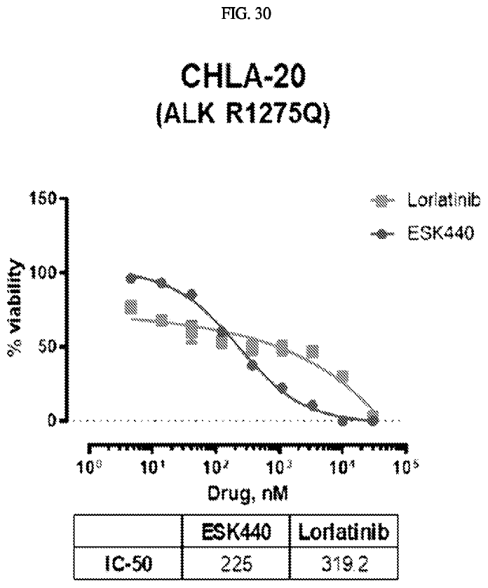

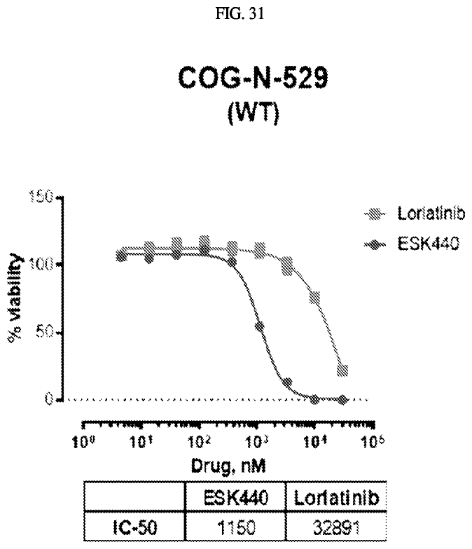

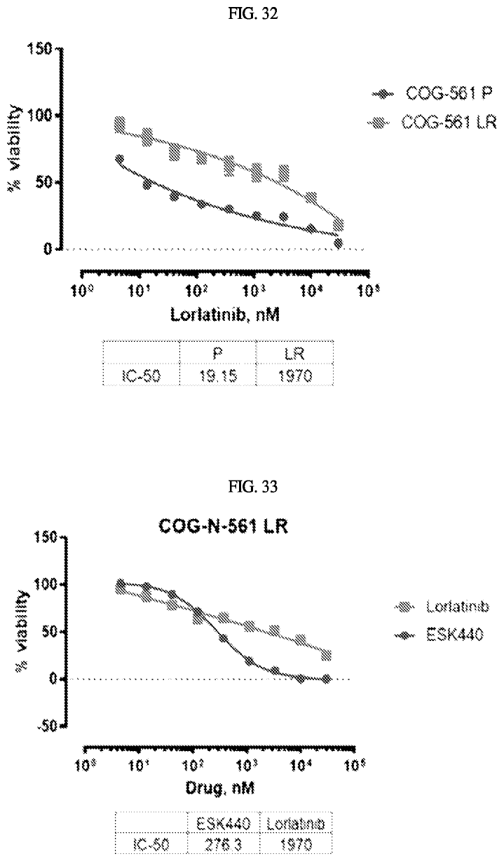

Lorlatinib (an ALK and ROS1 inhibitor) is currently being tested in clinical trials for NB; however, as with many targeted therapies, resistance and relapse can occur with lorlatinib. Cell viability assays between ESK440 and lorlatinib were performed in several NB cell lines. As shown in FIGS. 28-30, lower IC₅₀ values were achieved in some cell lines-NB-1643, CHLA-20, and COG-N-529-suggesting that ESK440 would achieve a better therapeutic response in ALK mutated NB. Also, a lorlatinib-resistant COG-561 cell line was created and treated with increasing doses of ESK440 (FIGS. 31 and 32). Results from this experiment showed that the lorlatinib-resistant cell line remained sensitive to treatment with ESK440. These data suggest that ESK440 would have efficacy in NB patients that have developed a resistance to lorlatinib.

Having now fully described the compounds, compositions, the methods herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

What is claimed is:

1. A method of treating neuroblastoma in a subject in need thereof, the method comprising administering to the subject a therapeutically effective amount of (S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide (ESK440).

2. The method of claim 1, wherein the neuroblastoma is characterized as having a genetic aberration of focal adhesion kinase (FAK), anaplastic lymphoma kinase (ALK), and/or N-myc proto-oncogene protein (MYCN).

3. The method of claim 1 or 2, wherein the neuroblastoma is an ALK-mutated neuroblastoma, a MYCN-amplified neuroblastoma, and/or a FAK-overexpressed neuroblastoma.

4. The method of claim 3, wherein the neuroblastoma is an ALK-mutated neuroblastoma comprising a F1275 mutation, an ALK F1245 mutation, or an ALK F1174 mutation.

5. The method of any one of claims 2-4, wherein the neuroblastoma is a MYCN-amplified neuroblastoma.

6. The method of claim 5, wherein the neuroblastoma is a FAK-overexpressed neuroblastoma.

7. The method of any one of claims 1-6, wherein the subject is aged 2 years to less than 12 years.

8. The method of any one of claims 1-6, wherein the subject is aged 12 years to less than 22 years.

9. The method of any one of claims 1-6, wherein the subject is aged 22 years or more.

10. The method of any one of claims 1-9, comprising administering ESK440 to the subject in combination with one or more anticancer agents to the subject.

11. The method of claim 10, wherein the anticancer agent is one or more of a chemotherapeutic agent, an immune checkpoint inhibitor, or radiation therapy.

12. A method of treating a subject having neuroblastoma, the method comprising:
(a) determining whether an ALK mutation, MYCN amplification, and/or FAK overexpression is present or absent in a biological sample taken from the subject; and
(b) administering a therapeutically effective amount of ESK440 to the subject if the ALK mutation, MYCN amplification, and/or FAK overexpression is present in the biological sample.

13. A method, comprising administering a therapeutically effective amount of ESK440 to a subject in need thereof, wherein:
(a) the subject has neuroblastoma; and
(b) the neuroblasoma is characterized as having an ALK mutation, MYCN amplification, and/or FAK overexpression.

14. A method of identifying whether a subject having neuroblasoma as a candidate for treatment with ESK440, the method comprising:
(a) determining whether an ALK mutation, MYCN amplification, and/or FAK overexpression is present or absent in a biological sample taken from the subject; and
(b) identifying the subject as being a candidate for treatment if the ALK mutation, MYCN amplification, and/or FAK overexpression is present; or
(c) identifying the subject as not being a candidate for treatment if the ALK mutation, MYCN amplification, and/or FAK overexpression is absent.

15. A method of predicting treatment outcome in a subject having neuroblasoma, the method comprising determining whether an ALK mutation, MYCN amplification, and/or FAK overexpression is present or absent in a biological sample taken from the subject, wherein:
(a) the presence of the ALK mutation, MYCN amplification, and/or FAK overexpression in the biological sample indicates that administering ESK440 to the subject will likely cause a favorable therapeutic response; and
(b) the absence of the ALK mutation, MYCN amplification, and/or FAK overexpression in the biological sample indicates that administering ESK440 to the subject will likely cause an unfavorable therapeutic response.

16. The method of any one of claims 12-15, wherein the neuroblasoma is characterized as having an ALK mutation.

17. The method of any one of claims 12-16, wherein the neuroblasoma is characterized as having MYCN amplification.

18. The method of any one of claims 12-17, wherein the neuroblasoma is characterized as having FAK overexperession.
